(19) United States
(12) Patent Application Publication
(10) Pub. No.: US 2016/0045257 A1
(43) Pub. Date: Feb. 18, 2016

(54) METHOD FOR SELECTION AND TREATMENT OF HYPERTENSIVE PATIENTS WITH RENAL DENERVATION

(71) Applicant: Ablative Solutions, Inc., Fair Haven, NJ (US)

(72) Inventors: Tim A. Fischell, Kalamazoo, MI (US); David R. Fischell, Fair Haven, NJ (US); Vartan Ghazarossian, Menlo Park, CA (US)

(21) Appl. No.: 14/459,524

(22) Filed: Aug. 14, 2014

(51) Int. Cl. A61B 18/18; A61N 7/00

(52) U.S. Cl. CPC A61B 18/18; A61N 7/00; A61N 2007/003; A61B 2018/00577

(57) ABSTRACT

Systems and methods are disclosed to identify and treat hypertensive patients most likely to benefit from a renal denervation procedure. The method can include identifying one or more signals that patient's hypertension is sympathetically driven; and performing a renal denervation procedure to denervate the sympathetic nerves located near at least one of the patient's renal arteries.

### FIELD OF THE INVENTION

This invention relates in some aspects to systems and methods to treat hypertension and other pathological states heralded by sympathetic nerve over-activity.

### BACKGROUND OF THE INVENTION

Since the 1930s it has been known that injury or ablation of the sympathetic nerves in or near the outer layers of the renal arteries can substantially reduce high blood pressure.

Over the last few years, numerous “minimally invasive” devices have been developed to perform renal sympathetic denervation (RDN). These devices include energy delivery devices that employ radiofrequency, such as Symplicity™ (Medtronic), EnligHTN™ (St. Jude Medical), and the PARADISE® (Recor) ultrasonic catheter system, among others. In addition Ablative Solutions has also developed a chemical renal denervation (RDN)-based Perivascular Tissue Ablation Catheter (PTAC) now FDA cleared as the Peregrine™ device (Fischell et al., in U.S. Patent Pub. Nos. 2013/0053821, 2013/0053792, and 2013/0053822, each of which are incorporated by reference in their entireties). Other approaches involving “non-invasive” methods, such as externally focused ultrasound are also being tested.

There are potential patient risks associated with the treatment of the renal arteries with surgery, or the above cited devices to perform renal denervation. Because of this, it can be advantageous to identify a group of patients for whom renal denervation is indicated, for example, those whose blood pressure is above the target despite medical therapy. This group of patients, among others, can be at increased risk for cardiovascular complications and death due to inadequate control of their hypertension and therefore can be justifiably considered for treatment with an invasive procedure.

One major challenge in identifying the population for treatment with renal denervation is the inaccuracy of office blood pressures. Conventional blood pressure (BP) measurements, performed in a medical setting (e.g., “office-based” BP) are not always accurate due to “white-coat hypertension” and therefore in some cases cannot be exclusively relied upon to diagnose patients whose blood pressure is truly refractory to medications, and who do not suffer from secondary hypertension due to a condition such as renal artery stenosis, fibromuscular dysplasia, Cushing's syndrome, pheocromocytoma, hyperaldosteronism, hyperthyroidism, carcinoid syndrome, aortic coarctation, etc. For patients suffering from secondary hypertension, the primary treatment is to address the underlying disease. Furthermore, office-based blood pressures occur at a single point in time or closely spaced intervals, even if multiple measurements are taken. Because blood pressure fluctuates throughout the day based on circadian rhythm, activity levels, food ingestion, dietary sodium, emotional states, timing of antihypertensive medications, and other factors, it can be challenging to use a simple office-based measurement of blood pressure as an accurate reflection of the patient's average blood pressure or even typical blood pressure.

Furthermore, it may be the case that not all patients with refractory essential hypertension will benefit from renal denervation. Indeed, some clinical trials of renal denervation suggest that 20-30% of patients are “non-responders.” Not to be limited by theory, but based on the mechanism of action of renal denervation, some patients who have a high level of sympathetic activation could have a higher likelihood of responding to renal denervation. However, the level of sympathetic activation is not typically measured in the clinical setting.

In some embodiments, the strategy of selecting the most appropriate target patients for renal denervation, therefore, involves identifying patients with essential hypertension (and ruling out etiologies of secondary hypertension), patients who: (1) have blood pressure that is consistently elevated and 2) who have true evidence of elevated sympathetic activity.

To overcome the limitations of office blood pressure, the following methods may be used, in some embodiments, to confirm consistent elevation of blood pressure, after secondary causes of hypertension are ruled out:
1. Elevated nocturnal ambulatory BP (ABPM) readings (e.g., greater than a mean SBP of 125 mmHg and/or a mean DBP>75 mmHg), and/or
2) High 24-hour ABPM measurement showing mean systolic ABPM reading(s) (e.g., >140 mmHg).

To date, one of the only well described methods for predicting the patients who will respond to RDN is related to assessing the patient's severity of hypertension, e.g., the patients with very high BP on many antihypertensive medications appear more likely to respond to RDN than those patients with lower BP on fewer medications. Unfortunately, this rudimentary and unrefined method for selecting patients still leaves a significant number of “non-responders” who are treated, and subsequently have no significant BP lowering while undergoing the additional risks associated with the RDN procedure. In addition it does not easily allow one to select and effectively treat those patients with moderate hypertension who might achieve an excellent clinical BP lowering response to RDN including those on one or two medications who may achieve better blood pressure control with RDN compared to additional drug treatment.

The direct measurement of renal sympathetic nerve activity with sensors that could be placed in the vicinity of the renal nerves, for example, has been proposed, the nerve sensing catheter described by Fischell et al in U.S. patent application Ser. No. 14/063,907 filed on Oct. 25, 2013, and hereby incorporated by reference in its entirety. This methodology can be invasive and not yet established as a predictive screening procedure. In addition, while measuring a change in nerve activity is feasible, one may not be able to readily ascertain patient independent nerve activity levels indicative of sympathetically driven essential hypertension (SDEH).

Sympathetic neurons emanate from the central nervous system. Innervation of the adrenal medulla by sympathetic neurons causes catecholamines (e.g., epinephrine, norepinephrine) to be released into the circulation and to function as hormones whereas sympathetic neurons innervating other organs, including the kidneys, release norepinephrine into their synapses, where it functions as a neurotransmitter. Because sympathetic activation affects multiple organs, evidence of heightened sympathetic activity affecting the kidney may be gleaned from evidence of increased sympathetic activity in other end-organs.

In some embodiments, systems and methods are discussed using one or more available non-invasive measures to better predict which patients have sympathetically-driven essential hypertension (SDEH), and thus allow a more appropriate patient selection, and enrich the therapeutic yield of treatment of hypertensive patients with RDN. Reliable predictors of BP response to renal denervation can be very valuable in order to select the appropriate patients for the interventional approach and to limit exposure of inappropriate patients to needless risk. Furthermore, reliable predictors will optimize the allocation of healthcare resources.

These same methods may apply for the appropriate selection of patients for other therapeutic uses of RDN, such as treatment of patients with congestive heart failure, atrial fibrillation, sleep apnea, glucose intolerance (including diabetes mellitus), etc.

### SUMMARY OF THE INVENTION

As suggested above, whereas neither office-based BP measurements nor the mean 24-hour ambulatory BP appear to provide adequate means to select patients who would respond to RDN treatment, there are a number of tests that may be more useful for patient selection. These tests or measures when performed and applied may help to discriminate, either alone or in combination patients in whom essential hypertension is driven by an inappropriate level of renal sympathetic nerve activity from those in whom it is not. The patients with sympathetically-driven hypertension are expected to be more likely to derive therapeutic benefit from RDN.

The methods described herein discuss the use of more specific measures that can be predictive of sympathetic nerve hyperactivity, and used either alone or in combination, one with another, to better predict those patients who are likely to achieve clinical benefit by the use of a renal denervation.

The specific tests that are proposed in this methodology include, but may not be limited to the following tests and measurements that are suggestive or predictive of increased sympathetic nerve activity:
1. Measurements from “first catch” (AM) urine sampling of urine norepinephrine levels to identify sympathetically driven essential hypertension. For example: the morning “catch” would be elevated if the total norepinephrine is, e.g., >20 mcg. The actual threshold could be normalized to GFR (kidney filtration rate) or may be patient-specific with potential threshold levels between 10 mcg and 50 mcg in some cases.
2. Nocturnal Ambulatory Blood Pressure >125 mmHg.
3. Ambulatory BP reading(s) demonstrating “non-dipping,” wherein nocturnal (mean) SBP and DBP ABPM is <10% reduced compared with the daytime mean SBP and DBP ABPM, respectively or Ambulatory BP reading(s) demonstrating “reverse dipping,” wherein nocturnal (mean) SBP and DBP ABPM is higher than daytime mean SBP and DBP ABPM, respectively.
4. Elevated 24-hour urine norepinephrine level(s) (e.g., >80 mcg/24 hours).
5. Elevated morning upright ratio of serum aldosterone to renin (e.g., >30).
6. Low Baroreflex sensitivity (BRS) [e.g., <3.0 MS/mmHg] and/or low heart rate variability (Standard Deviation of the Normal to Normal R-R interval, SDNN) [e.g., <70 sec] on Holter Monitoring.
7. Elevated plasma renin activity (PRA) [peripheral venous] (e.g. >3.0 ng/mL/hour if Na replete and >10.8 ng/mL/hour if Na depleted).
8. Elevated plasma metanephrine, epinephrine and/or norepinephrine levels, for example, above the thresholds below:

| | |
|---|---|
| Free metanephrine | >0.5 nmol/L |
| Free normetanephrine | >0.90 nmol/L |
| Free epinephrine | Supine >111 pg/mL |
| | Standing >141 pg/mL |
| Norepinephrine | Supine >750 pg/mL |
| | Standing >1,700 pg/mL |

9. Increased muscle sympathetic nerve activity measurements (MSNA) (e.g., greater than the published mean value in hypertensive patients within age group (i.e., burst rate/min >23.5, >28.6, and >39.4 in patients <=30 years, 31-50 years, and >51 years respectively or burst incidence [bursts/100 heartbeats]>34.8, >43.7, and >62.6 in patients <=30 years, 31-50 years, and >51 years, respectively. More stringent criteria such as top 25^{th} percentile based on normative values for hypertensive patients may also be applied.
10. Random urine metanephrine/creatinine ratio: >154 mcg/g creatinine and/or other blood or urinary hormones or their respective metabolites.

Finally, it is envisioned that sympathetically driven essential hypertension can be identified by measurement of renal nerve sympathetic activity by direct nerve activity recording from a catheter placed within the renal artery and/or electrodes placed through the renal artery wall into the adventitial and/or periadventitial space, wherein the renal sympathetic nerves lie.

These tests and measurements may be conducted serially as is shown in FIG. 1 or more than one test may be run at a time. In some embodiments, the tests and measurements can be performed after ruling out causes of secondary hypertension, which would involve treating the underlying cause of the secondary hypertension. While any one of the above could be an indication of sympathetically driven essential hypertension (SDEH), it is also envisioned that two or more could be required to identify a patient with SDEH.

Thus, an object of this invention is a method for treating sympathetically driven essential hypertension (SDEH) by a first step of identifying SDEH followed by performance of one, two, or more Renal Denervation (RDN) procedures. This method is applicable to either energy or chemical RDN procedures.

Still another object of the invention is to use specific combinations of two or more measures that indicate increased sympathetic nerve activity to screen patients prior to treatment with renal denervation.

Still another object of the invention is to use this methodology to decrease the incidence of non-response to therapeutic renal denervation for the treatment of hypertension and therefore improve therapeutic efficacy and minimize patient exposure to an invasive procedure that may bear health risks.

Still another object of the invention is to use this methodology to decrease the incidence of non-response to therapeutic renal denervation for the treatment of left ventricular hypertrophy.

Still another object of the invention is to use this methodology to decrease the incidence of non-response to therapeutic renal denervation for the treatment of congestive heart failure.

Still another object of the invention is to use this methodology to decrease the incidence of non-response to therapeutic renal denervation for the possible use to retard progression of chronic kidney disease.

Still another object of the invention is to use this methodology to decrease the incidence of non-response to therapeutic renal denervation for the treatment of atrial fibrillation.

Still another object of the invention is to use this methodology to decrease the incidence of non-response to therapeutic renal denervation for the treatment of sleep apnea.

Still another object of the invention is to use this methodology to decrease the incidence of non-response to therapeutic renal denervation for the treatment of glucose intolerance or diabetes mellitus.

Still another object of the invention is to avoid treating non-responding patients, and therefore, save health care expenses.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a flow chart showing an example of a serial process for identifying and treating sympathetically driven hypertension in a human patient.

FIG. 2 is a schematic view of the distal portion of a Perivascular Tissue Ablation Catheter (PTAC) in its open position as it would be manually expanded for delivery of an ablative agent into the peri-vascular space. This can be as disclosed, for example, in U.S. patent application Ser. No. 13/342,521, which is hereby incorporated by reference in its entirety.

### DETAILED DESCRIPTION

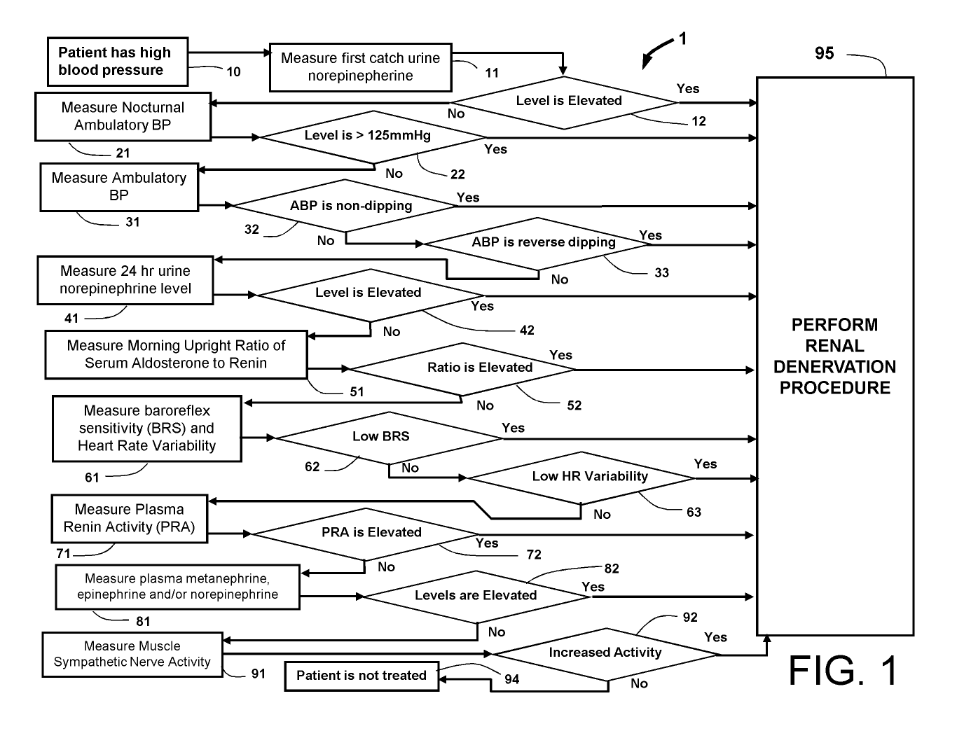

FIG. 1 is a flow chart showing an example of a serial process 1 for identifying and treating sympathetically driven hypertension in a human patient. A patient enters the process in step 10 which assumes they already have been identified as having high blood pressure (hypertension). The process begins with step 11 where the first catch urine of the morning is tested for norepinephrine levels which if they are elevated as determined in step 12, identifies the patient as having Sympathetically Driven Essential Hypertension (SDEH) and the patient proceeds to step 95 where a renal denervation procedure is conducted. For example: the morning catch could be elevated if the total norepinephrine is >10, 15, 16, 17, 18, 19, 20, 21, 22, 23, 24, 25, 30, 35, 40, 45, 50, or more mcg. The actual threshold could be normalized to GFR (kidney filtration rate) or may be patient specific with potential threshold levels between 10 mcg and 50 mcg.

If the level of morning catch of urine norepinephrine is not elevated, then the process 1 step 12 proceeds to step 21. Nocturnal Ambulatory Blood Pressure above 125 mmHg (or above 120 mmHg or 130 mmHg in some embodiments) will identify the patient as having SDEH and they proceed to step 95. If not the process 1 proceeds to step 31 to measure Ambulatory blood pressure throughout the day and a patient is identified with SDEH if Ambulatory BP reading(s) demonstrate “non-dipping,” (step 32) wherein nocturnal (mean) SBP and DBP ABPM is <10% reduced compared with the daytime mean SBP and DBP ABPM, respectively or Ambulatory BP reading(s) demonstrate “reverse dipping,” (step 33) wherein nocturnal (mean) SBP and DBP ABPM is higher than daytime mean SBP and DBP ABPM, respectively. If SDEH is identified then the process 1 proceeds to step 95.

If not then the process 1 continues with the measurement of 24 hour urine norepinephrine levels in step 41. If in step 42 24-hour urine norepinephrine level(s) are elevated (e.g., >80 mcg/24 hours, or >60, 65, 70, 75, 80, 85, 90, 95, or 100 mcg/24 hours), then the process 1 proceeds to step 95.

If not then in step 51 the process 1 includes the measurement of morning upright ratio of serum aldosterone to renin which if in step 52 is seen as elevated (e.g., >15, 20, 25, 30, 35, 40, 45, or 50) identifies the patient as having SDEH and the process 1 proceeds to step 95.

If the morning upright ratio of serum aldosterone to renin is not elevated the process 1 continues with step 61 to measure baroreflex sensitivity (BRS) and heart rate variability. Either low baroreflex sensitivity (BRS) as seen in step 62 (e.g., <3.0 ms/mmHg, or <4.0, 3.5, 2.5, or 2.0 ms/mmHg in some embodiments) and/or low heart rate variability seen in step 63 (Standard Deviation of the Normal to Normal R-R interval, SDNN) [e.g., <75, 74, 73, 72, 71, 70, 69, 68, 67, 66, 65, or less msec] on Holter Monitoring will identify the as having SDEH and the process 1 proceeds to step 95. If neither is low then the process 1 proceeds to step 71.

Step 71 is the measurement of plasma renin activity (PRA). If in step 72 is determined that peripheral venous PRA is Elevated (e.g. >3.5, 3.4, 3.3, 3.2, 3.1, 3.0, 2.9, 2.8, 2.7, 2.6, 2.5 ng/mL/hour, or more if Na repleted and >10, 10.5, 10.6, 10.7, 10.8, 10.9, 11.0, 11.5 ng/mL/hour, or more if Na depleted) then the patient is identified as having SDEH and the process 1 proceeds to step 95. If neither is low then the process proceeds to step 81.

In step 81 the process 1 includes the measurement of plasma metanephrine, epinephrine and/or norepinephrine levels. If any of these are elevated, for example above the thresholds below step 82 identifies the patient as having SDEH and the process 1 proceeds to step 95.

| | |
|---|---|
| Free metanephrine | >0.5 nmol/L (or >0.3, 0.4, 0.6, |
| | 0.7, 0.8, 0.9, 1.0, or more |
| | nmol/L in some embodiments) |
| Free normetanephrine | >0.90 nmol/L (or >0.80, 0.85, |
| | 0.95, 1.00, 1.05, or 1.10 nmol/L) |
| | in some embodiments) |
| Free epinephrine | Supine >111 pg/mL (or >100, |
| | 105, 106, 107, 108, 109, 110, |
| | 111, 112, 113, 114, 115, 120, |
| | or more pg/mL in some |
| | embodiments) |
| | Standing >141 pg/mL (or |
| | <130, 135, 136, 137, 138, |
| | 139, 140, 142, 143, 144, 145 pg/mL, |
| | or more in some |
| | embodiments) |
| Norepinephrine | Supine >750 pg/mL (or >700, |
| | 725, 775, or 800 pg/mL in |
| | some embodiments) |
| | Standing >1,700 pg/mL (or |
| | >1,550, 1,600, 1,650, 1,750, |
| | or 1,800 pg/mL in some |
| | embodiments) |

If none of the levels are elevated, the process 1 proceeds to step 91.

In step 91 sympathetic nerve activity measurements are conducted. In step 92 the levels are assessed and the patient is identified with SDEH and proceeds to step 95 if there is increased muscle sympathetic nerve activity measurements (MSNA) (e.g., greater than the published mean value in hypertensive patients within age group (e.g., burst rate/min >23.5, >28.6, and >39.4 in patients <=30 years, 31-50 years, and >51 years respectively or burst incidence [bursts/100 heartbeats]>34.8, >43.7, and >62.6 in patients <=30 years, 31-50 years, and >51 years, respectively. More stringent criteria such as top 35, 30, 25, 20, 15, 10, or other percentiles based on normative values for hypertensive patients may also be applied. If there is no increased activity determined, the process 1 proceeds to step 94 and the patient is not treated with renal denervation (step 95)

Other tests that can be added to this flow chart include, but are not limited to:
1. Random urine metanephrine/creatinine ratio: >140, 145, 150, 154, 155, 160, 165 mcg/g, or more creatinine and/or other blood or urinary hormones or their respective metabolites; and/or
2. Measurement of renal nerve sympathetic activity by direct nerve activity recording from a catheter placed within the renal artery and/or electrodes placed through the renal artery wall into the adventitial and/or periadventitial space, wherein lie the renal sympathetic nerves. In some embodiments, the activity is >10%, 20%, 30%, 40%, 50%, 60%, 70%, 80%, 90%, 100%, or more greater than the normal sympathetic activity expected for a given patient.

While FIG. 1 shows the process 1 as a serial process with certain selected tests to identify SDEH first, it is clear that one could have a different order of such a serial process. It is also envisioned that more than one of these tests could be performed at the same time and the results allowing the identification of SDEH more quickly and with greater specificity. It is also envisioned that to further enhance the predictive accuracy for the identification of SDEH the final criteria could include a requirement that two, three, four or some other number of these tests that indicate SDEH must be positive before the process 1 proceeds to renal denervation in step 95.

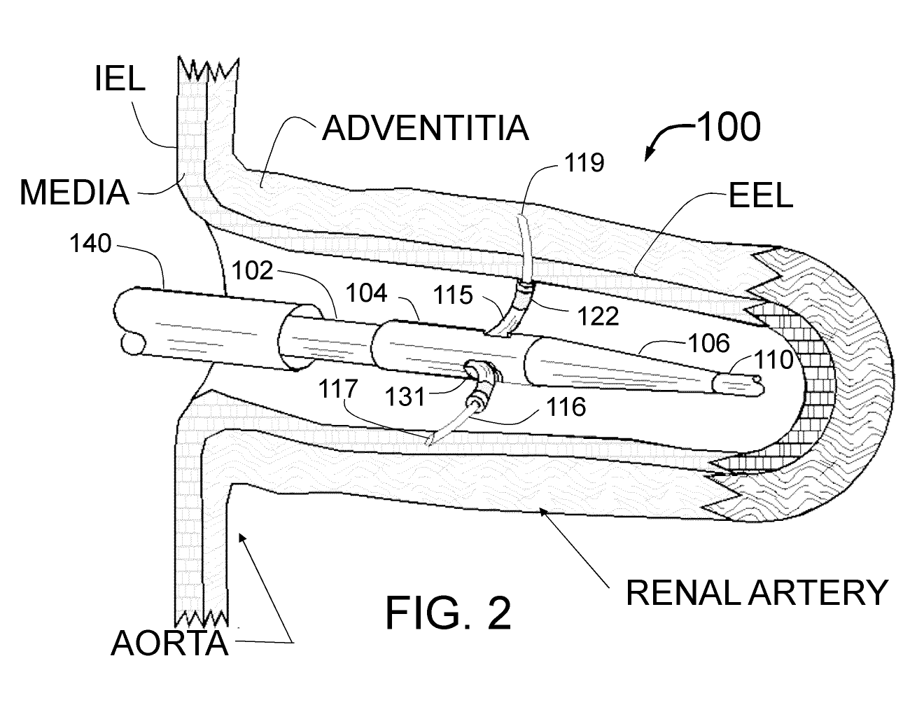

FIG. 2 is a schematic view of the distal portion of a PTAC 100 in its open position within a renal artery, showing an outer tube 102, outer tube extension 104 having distal openings 131 through which the guide tubes 115 with radiopaque markers 122 are advanced outward from the body of the PTAC 100. Also shown is the tapered section 106 and fixed guide wire 110. The injector tubes 116 with distal injection needles 119 and needle distal openings 117 are shown in their fully deployed positions. The openings 131 support the sides of the guide tubes 115 as the guide tubes 115 are advanced outward before the advancement of the injector tubes 16 with distal injector needles 119. The PTAC 100 of FIG. 2 has three guide tubes with the third tube hidden behind the catheter and not visible in this schematic view. Although the PTAC 100 of FIG. 2 has three guide tubes 115, it is envisioned that other embodiments could have as few as one or as many as eight guide tubes with an optimum number being three or four. A larger diameter target vessel might suggest the use of as many as 4 to 8 guide tubes 115 and injector tubes 116.

FIG. 2 shows the PTAC 100 with injector tubes 116 with distal injection needles 119 fully deployed to deliver an ablative fluid into the peri-vascular space within or outside of the adventitial of the renal artery. Ideally—the needle distal openings 117 at or near the distal end of the injection needles 119 should be positioned beyond the EEL and toward the outside of the adventitia as shown for the upper needle 119 in FIG. 10. The sympathetic nerves which are the target for renal denervation lie within the adventitia or within several millimeters outside of the adventitia. Specifically a distance of 2-4 mm beyond the Internal Elastic Lamina (IEL) is the appropriate position for the needle distal opening 117. If the sympathetic nerves are deeper, it is also envisioned that depths of 4 to 8 mm could be used. This is one embodiment of the invention disclosed in U.S. patent application Ser. No. 13/342,521, incorporated by reference in its entirety.

The PTAC of FIG. 2 is only one embodiment of a potential device for renal denervation. Other chemical and energy systems can also be used or modified for use with the systems and methods disclosed herein, including the Symplicity™ RF ablation system from Medtronic, the Bullfrog™ infusion catheter from Mercator, and the fluid injection and energy based catheters described by Fischell et al. in U.S. Pat. No. 8,740,849, as well as U.S. patent application Ser. Nos. 13/216,495, 13/294,439, 13/342,521, and 13/643,065, each of which are hereby incorporated by reference in their entireties.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “performing a renal denervation procedure” include “instructing the performing of a renal denervation procedure.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A method for the treatment of sympathetically driven essential hypertension comprising the steps of:
a. identifying one or more signals that patient's hypertension is sympathetically driven; and
b. performing a renal denervation procedure to denervate the sympathetic nerves located near at least one of the patient's renal arteries.

2. The method of claim 1 where the one or more signals to identify sympathetically driven hypertension comprises one or more selected from the group consisting of:
a. Elevated norepinephrine from first catch (AM) urine sampling;
b. Nocturnal Ambulatory Blood Pressure >125 mmHg;
c. Ambulatory BP reading(s) demonstrating “non-dipping,” wherein nocturnal (mean) SBP and DBP ABPM is <10% reduced compared with the daytime mean SBP and DBP ABPM, respectively;
d. Ambulatory BP reading(s) demonstrating “reverse dipping,” wherein nocturnal (mean) SBP and DBP ABPM is higher than daytime mean SBP and DBP ABPM, respectively;
e. Elevated 24-hour urine norepinephrine level;
f. Elevated morning upright ratio of serum aldosterone to renin;
g. Low baroreflex sensitivity;
h. Elevated plasma renin activity;
i. Elevated plasma metanephrine levels;
j. Elevated epinephrine levels;
k. Elevated plasma norepinephrine levels;
l. Increased muscle sympathetic nerve activity; and
m. Random urine metanephrine/creatinine ratio: >154 mcg/g creatinine.

3. The method of claim 1 where the measurement of blood hormones is used to identify sympathetically driven hypertension.

4. The method of claim 1 where measurement of blood hormones or their metabolites is used to identify sympathetically driven hypertension.

5. The method of claim 1 where measurement of urinary hormones is used to identify sympathetically driven hypertension.

6. The method of claim 1 where measurement of urinary hormones or their metabolites is used to identify sympathetically driven hypertension.

7. The method of claim 1 where the measurement of renal nerve sympathetic activity by direct nerve activity recording is used to identify sympathetically driven hypertension.

8. The method of claim 1 where RF ablation is used to perform the renal denervation procedure.

9. The method of claim 1 where ultrasound is used to perform the renal denervation procedure, delivered endovascularly or externally.

10. The method of claim 1 where injection of one or more chemicals are used to perform the renal denervation procedure.

11. The method of claim 1 where the renal denervation procedure is performed using a catheter from within the renal artery.
